(19) United States
(12) Patent Application Publication
(10) Pub. No.: US 2011/0251652 A1
(43) Pub. Date: Oct. 13, 2011

(54) NEURAL PROSTHETICS USING A CHEMICAL HARVESTING AND STIMULATION DEVICE

(75) Inventors: Jongyoon Han, Bedford, MA (US); Yong-Ak Song, Newton, MA (US); Amr Rabie, Cairo (EG); Rahul Sarpeshkar, Arlington, MA (US); Samuel J. Lin, Needham, MA (US)

(73) Assignees: BETH ISRAEL DEACONESS MEDICAL CENTER, INC., Boston, MA (US); MASSACHUSETTS INSTITUTE OF TECHNOLOGY, Cambridge, MA (US)

(21) Appl. No.: 13/083,014

(22) Filed: Apr. 8, 2011

Related U.S. Application Data
(60) Provisional application No. 61/322,025, filed on Apr. 8, 2010.

(51) Int. Cl. A61N 1/36; C12N 13/00

(52) U.S. Cl. 607/2; 435/173.1

(57) ABSTRACT

A neural prosthetic device is provided that includes one or more ion-selective membranes enabling electrically-controlled local modulation of ion concentrations around a nerve so as to achieve different excitability states of the nerve for electrical stimulation or inhibition of nerve signal propagation.

### PRIORITY INFORMATION

This application claims priority from provisional application Ser. No. 61/322,025 filed Apr. 8, 2010, which is incorporated herein by reference in its entirety.

### BACKGROUND OF THE INVENTION

The invention is related to the field of neural prosthetics, and in particular an electrochemical artificial nerve activation and inhibition technique, enabled by electrically-controlled local modulation of ion concentrations along the nerve

Conventional functional electrical stimulation (FES) aims to restore functional motor activity for patients with disabilities resulting from spinal cord injury (SCI) or neurological disorders by artificially stimulating the nerve. Among the many technical limitations of FES-related intervention in neurological diseases, the most crucial drawback is the lack of an effective, implantable technique for nerve signal simulation and conduction block for suppressing unwanted nerve signals.

Achieving cures for SCI patients requires progress both in scientific understanding of basic neurophysiology and engineering techniques for neural activation and modulation. FES has been associated with substantial therapeutic benefits of physical activity; it has been used to increase muscle bulk, improve cardiovascular performance, prevent and treat pressure ulcers, treat osteoporosis and joint contractures, control spasticity, and improve general well being; moreover, FES can be critical for recovery of neurological function and can be essential for maintenance of neural circuitry, should a cure be found. However, high-energy expenditure and the lack of a totally implantable FES system have limited its value and are among the various bioengineering reasons for the inability to create widely acceptable FES systems. Moreover, electrical stimulation that produces muscle contraction in humans also stimulates sensory nerves and pain receptors, causing pain. Therefore, reducing energy expenditure by lowering the electrical threshold not only increases battery life, but also reduces the patient's pain associated with the electrical stimulation. Aside from nerve activation techniques, the ability to suppress unwanted nerve signals would be of great potential value to not only neuroprosthetics but also various clinical situations. A nerve signal conduction blocking technique, which can arrest the propagation of action potentials in a graded, safe, and reversible fashion, could be applied to suppress nerve and/or motor activity that may include but are not limited to undesired sensation, such as pain, neuralagia, tinnitus, vertigo or deleterious motor activity, such as muscle hypertonicity, spasm, dystonias, chronic migraine, hyperhidrosis, blepharospasm, strabismus, Achalasia, neurogenic bladder, diabetic neuropathy, upper motor neuron syndrome, and/or spasticity.

Existing techniques such as pharmacological treatments or surgical interventions all have significant disadvantages. For instance, conventional surgical treatment of unwanted noxious painful stimuli may consist of nerve ablation, or permanent division of the nerve; when a nerve has both sensory and motor function, function may be permanently lost with nerve ablation. The use of high-frequency alternating current waveforms was previously reported as one potential technique for nerve conduction block. This technique has been shown to produce a quickly reversible nerve block under isolated conditions in frog, rat, cat and dog models. For instance, in the frog, a continuous sinusoidal or rectangular waveform at 3-5 kHz and amplitudes at 0.5-2 mAₚ₋ₚ allowed the most consistent block. However, no implant device has been demonstrated based on this approach so far.

### SUMMARY OF THE INVENTION

According to one aspect of the invention, there is provided a neural prosthetic device. The neural prosthetic device includes one or more ion-selective membranes enabling electrically-controlled local modulation of ion concentrations around a nerve so as to achieve different excitability states of the nerve for electrical stimulation or inhibition of nerve signal propagation.

According to another aspect of the invention, there is provided a method of performing active nerve stimulation or inhibition of nerve signal propagation. The method includes providing one or more ion-selective membranes. Also, the method includes electrically controlling local modulation of ion concentrations using the one or more ion-selective membranes around a nerve so as to achieve different excitability states of the nerve for electrical stimulation or inhibition of nerve signal propagation.

### BRIEF DESCRIPTION OF THE DRAWINGS

FIGS. 1A-1B are schematic diagrams illustrating an array of ion-selective electrodes in a flexible biocompatible plastic chip;

FIGS. 2A-2B are schematic diagrams illustrating a first embodiment of the invention;

FIGS. 3A-3D are graphs illustrating what occurs before and after Ca²⁺ depletion;

FIGS. 4A-4B are graphs illustrating the effects of K⁺ ion depletion on the nerve excitability;

FIG. 5 is a graph illustrating depletion of Ca²⁺ ions in Ringer's solution as a function of depletion time;

FIG. 6A-6B are schematic diagrams illustrating flexible neural stimulation devices formed in accordance with the invention;

FIGS. 7A-7C are schematic diagram illustrating a second embodiment of the invention;

FIGS. 8A-8B are graphs illustrating comparison of excitability without and with modulating the Ca²⁺ ion concentration

FIGS. 9A-9B is a schematic diagram and graph illustrating the effect of ion concentration modulation on the nerve signal blocking;

FIGS. 10A-10B are graphs illustrating the effect of cation depletion on the signal propagation along the sciatic nerve; and

FIGS. 11A-11B are graphs illustrating the effect of Ca²⁺ ion depletion on the tetany-like muscle twitching.

### DETAILED DESCRIPTION OF THE INVENTION

The invention describes an electrochemical artificial nerve activation and inhibition technique, enabled by electrically-controlled local modulation of ion concentrations along the nerve. In this technique, the concentration of ions is modulated around the nerve in-situ using an ion-selective membrane (ISM), in order to achieve different excitability states of the nerve for electrical stimulation, leading to either reduction of electrical threshold by up to approximately 40% or on-demand, reversible inhibition of nerve signal propagation. This low-threshold electrochemical stimulation technique would be used in an implantable neuroprosthetic device, while the on-demand nerve blocking could offer a novel intervention for chronic disease states caused by uncontrolled nerve activation, such as epilepsy and chronic pain stimuli.

A key component of the invention is the in-situ control of ion concentration via ion-selective electrode that can provide a novel mode of local nerve activation (excitatory) and inactivation (inhibitory) in a potentially very low-power, highly miniaturizable and efficacious fashion. This hybrid approach is tested using a sciatic nerve of an animal (e.g., frog), attached to the gastrocnemius muscle that can be surgically removed from the animal and placed on top of an array of ion-selective electrodes. Monovalent or divalent salt ions such as Na⁺, K⁺—, Ca²⁺ ions selective electrodes can be used to actively control the local ion concentration either by decreasing or increasing a specific ion concentration along the sciatic nerve fiber prior to electrical stimulation. The outcome of the subsequent electrical stimulation is quantitatively measured in two different ways. Firstly, the force induced is measured in the muscle using a force transducer. Secondly, electromyography (EMG) is employed directly on the muscle to measure the muscle response. Various electrical/chemical stimulus conditions are employed and the resulting force/EMG signals are measured. One is able to determine how far the electrical threshold value required for stimulation can be decreased by modulating the local Ca²⁺ ion concentration.

With the modulation of the K⁺ and Na⁺ ion concentration, it will be determined if one can reversibly turn “on and off” the signal propagation along the nerve. Another expected outcome of the invention is that the activity in the muscle (in terms of force, or other quantitative characteristics from EMG) can potentially be controlled with higher degree of resolution and/or dynamic range, as a function of ion concentration parameter compared with the case of pure electrical stimulation.

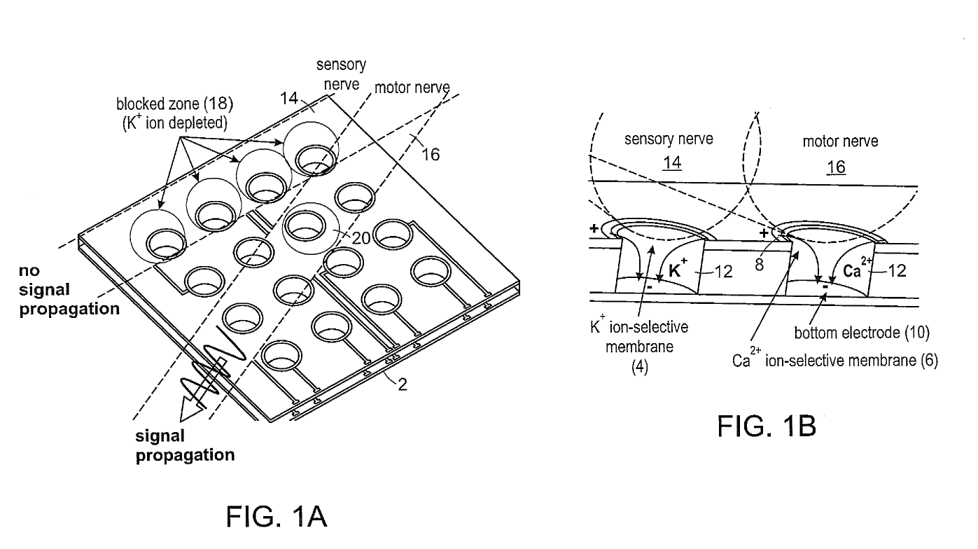

Using the concepts of the invention one can build a microfabricated implant device with an array of ion-selective electrodes in a flexible biocompatible plastic chip format, as shown in FIGS. 1A-1B. FIG. 1A shows flexible ion-selective microelectrode array device 2 in a 4×4 array with two different ion-selective membranes 4, 6 for enhanced or inhibited electrical stimulation based on the modulation of ion (Ca²⁺ and K⁺ ions) concentrations. The flexible microelectrode array device 2 includes bio-compatible material that can be used in a planar or folded configuration. FIG. 1B shows the cross-sectional view of the flexible ion-selective microelectrode array device 2. Ca²⁺ and K⁺ ion-selective membranes 4, 6 are sandwiched between a ring-type electrode 8 on the top and a circular type electrode 12 on the bottom layer 10. The current applied across the ion-selective membrane to modulate the ion concentration (˜100 nA to 1 μA) is approximately one or two orders of magnitude less than the typical electrical threshold value required for stimulation (˜10 μA).

Each of the ion-selective electrodes 4,6 can be individually controlled to create a hypersensitive zone in a motor nerve 16 for an electrical stimulation 18 and an inhibited/blocked zone 20 in the neighboring sensory nerves 14 to block the nerve signal, minimizing the pain induced by the electrical stimulation. This flexible device 2 can be used in highly space-constrained regions of the body such as the orbit of the eye or the face. This electrode arrangement is bipolar, however, in other embodiments of the invention the electrode arrangement can be tripolar which is further discussed hereinafter.

A unique characteristic of the inventive device is its ability to modulate neural activity, either locally stimulating or blocking nerve impulses by changing the Ca²⁺ or K⁺ ion concentration in or around the nerve. It is the first time that an ion-selective microelectrode array on flexible substrate is used as a neural interface.

It is well known that a relatively small change in the potassium ion concentration can increase the membrane potential from its resting value of −74 mV by 24 mV to reach a neuron firing initiation potential while other ions such as Na⁺ and Cl⁻ have no significant impact. In fact, potassium solutions as dilute as 10 mM are commonly used to depolarize neurons. Another ion associated with the excitability of peripheral nerve in neurology, both experimental and clinical, is the ionized calcium in the bathing solution. The available data for A-fibers in the sciatic nerves of the frog showed that the threshold intensity of direct current decreased markedly when the concentration of ionized calcium was below about 0.8 mM while a negligible change occurs in the range from 1 to 5 mM. A similar result has been found in the case of an isolated giant axon of the squid. However, the concentration range within which the changes occur is much higher for nerves from squid than for those from frog (10-70 mM in artificial sea water). The generality of this phenomenon has also been observed in the blood of humans with hypoparathyroid disease. A correlation between the measured excitability of the ulnar nerve and the concentration of calcium has been reported. In the lower concentration range, the nerve becomes more excitable and below 0.3 mM, alpha fibers in the sciatic nerve of frogs may become spontaneously active. If the concentration of calcium chloride is increased to 10-15 mM, then the threshold for excitation increases again.

Based on this significant role of potassium and calcium ions in neural processes, one can develop a technique to actively modulate the ion concentration around the nerve in a highly local manner, to achieve higher excitability when stimulating the nerve electrically or to initiate inhibitory state as the opposite effect. To modulate the ion concentration around the nerve, the ion-selective electrodes (ISE) are used. These electrodes include an ion-selective membrane at the tip and an electrode inside the tip. The membranes can be made using an ion selective agent such as an ionophore to increase the permeability of the selective layer in a plasticized amorphous polymer matrix such as polyvinyl chloride. To test the viability of modulating ion concentration with ISE for an electrical stimulation, an array of the wire electrodes is built and placed an ISE underneath one wire electrode which served for both ion depletion and electrical stimulation.

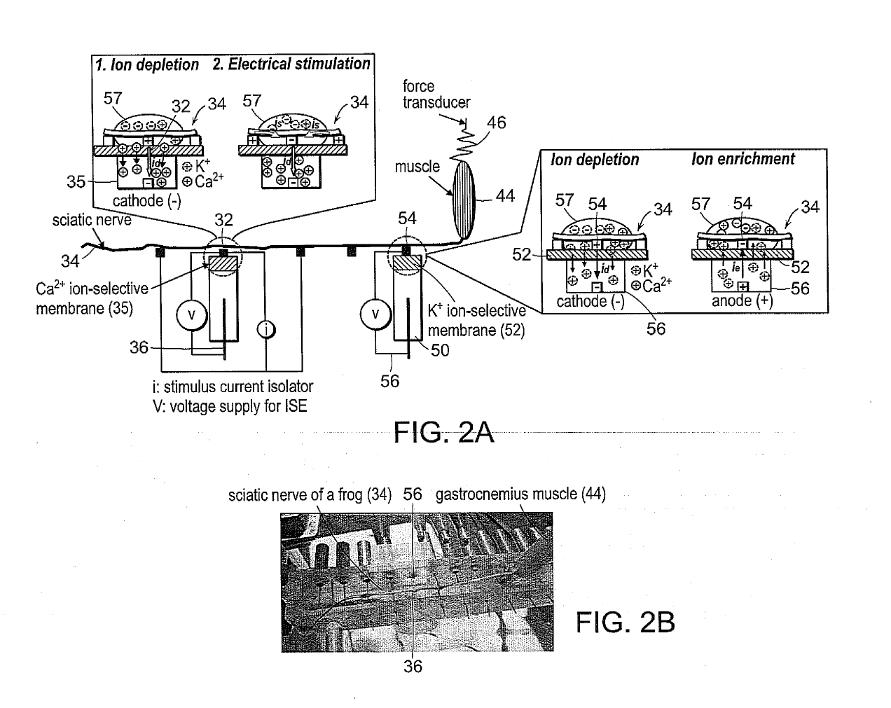

An embodiment of the invention is shown schematically in FIG. 2A. In this setup 30, two modes of operation, ion depletion and stimulation, are implemented. First, an ion depletion current i_{d} is applied across the ion-selective membrane 35 between the wire electrode 32 directly underneath the nerve 34 and the cathode 36 inside the tubing 37, as shown in inset 40. After depleting the ions at a given current i_{d} (ion depletion current id should be smaller than the threshold value for electrical stimulation is) for duration t, the on depletion current i_{d} is turned off and applied an electrical stimulus current iₛ into the nerve 34 while the cathode 36 was floating in the second step. The cathode 36 and electrode 32 are coupled to a voltage source V for ISE and a current source I is coupled to the electrode 32 as a stimulus current isolator. A cathode 56 is used to provide nerve blocking under K⁺ ion depletion and enrichment. An electrode 54 is positioned underneath the nerve 34 to establish K⁺ ion depletion or enrichment. The electrode 54 is positioned on a K⁺ ion-selective membrane 52. The tubing 50 covers the cathode 56. The cathode 56 and electrode 54 are coupled to a voltage source V. To test this electrochemical stimulation technique, a sciatic nerve 34 of a bull frog with its gastrocnemius muscle 44 was removed and placed on the electrode array 30, as shown in FIG. 2B. The end of the gastrocnemius muscle 44 is attached to a force transducer 46 via string. The standard Ringer's solution 57 is used to wet the nerve 34 and the muscle 44 with. The signal of the force transducer 46 was recorded with a data acquisition unit and analyzed with processing software. In addition to the muscle contraction force, the compound action potential is measured with an amplifier and a PC-based oscilloscope.

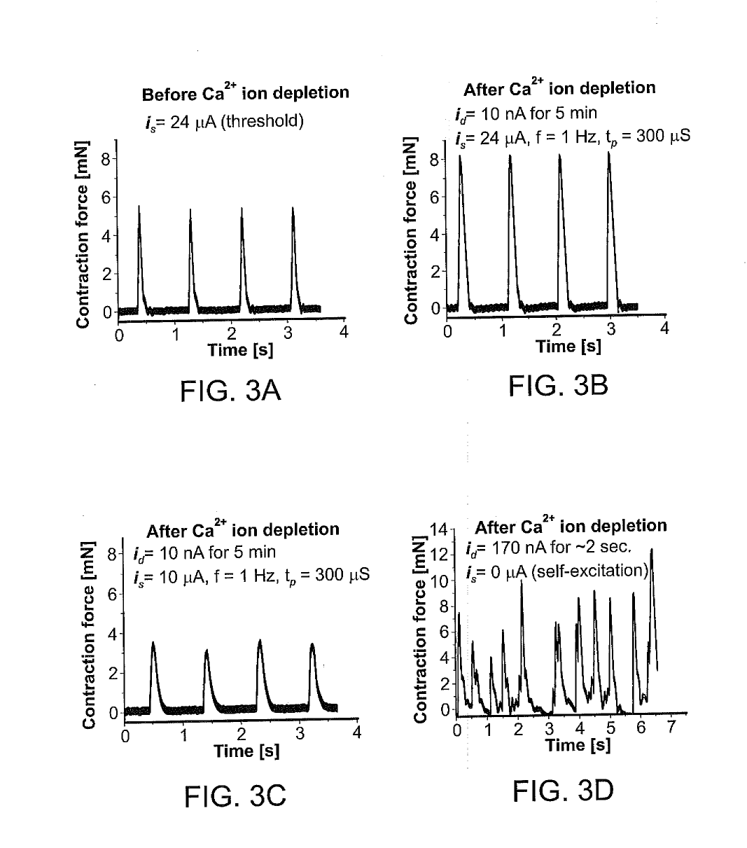

A typical electrical stimulus used in this case was iₛ=24 μA at a pulse width of tₚ=300 μS and a pulse frequency of f=1 Hz. Using a Ca²⁺ ion-selective electrode on the sciatic nerve of a frog, a 50% higher muscle contraction force is achieved with the same current pulse, compare FIG. 3A to FIG. 3B, and decreased the electrical current threshold value to initiate a muscle contraction by ˜50% down to iₛ=10 μA when Ca²⁺ ions were depleted locally at the site of the electrical stimulation for 5 min., as shown in FIG. 3C.

To deplete the Ca²⁺ ions, a depletion current of i_{d}=10 nA is applied across an ion-selective membrane for 5 min., and then applied stimulation electrical pulses iₛ directly thereafter. It was even possible to elicit a spontaneous activity of the nerve without applying any external electrical pulse by simply depleting the Ca²⁺ ions, as shown in FIG. 3D. An ion concentration below 0.3 mM can initiate such a spontaneous excitation of the nerve. However, the amplitude of muscle contraction was not uniform compared to the electrically elicited muscle contractions. This result in FIG. 3D suggests that one can achieve fast ion depletion simply by increasing the ion depletion current i_{d} across the ion-selective membrane (from 5 min. at i_{d}=10 nA, as shown in FIGS. 3B and 3C, down to ˜2 sec at i_{d}=170 nA). The depletion current was still ˜100 times smaller than the normal threshold value.

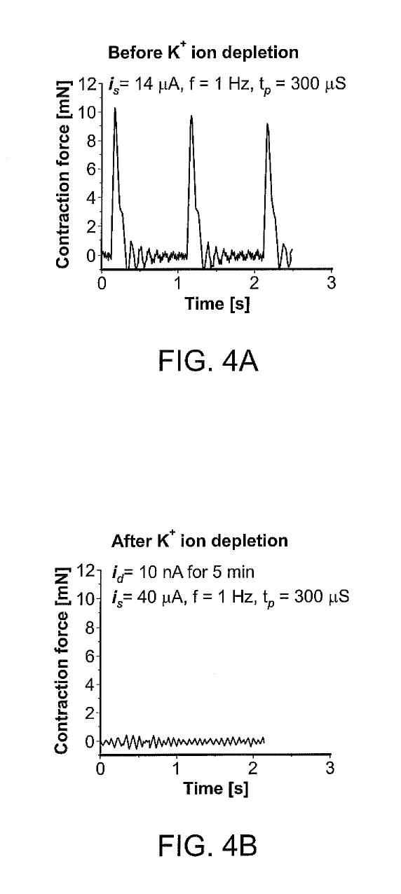

To avoid the tetany occurring below 0.3 mM, one could most likely have used less depletion time than 2 sec. to obtain a hypersensitive state of the nerve for electrical stimulation. In the case of a K⁺ ion selective electrode, the opposite effect of the ion depletion on the electrical stimulation is observed. One could completely inactivate the nerve for an electrical stimulus by depleting the K⁺ ions from the nerve and its surroundings. No muscle response was recorded even at higher electrical pulses iₛ over 40 μA after depleting K⁺ ions for 5 min. with i_{d}=10 nA across the ion-selective membrane, as shown in FIGS. 4A-4B.

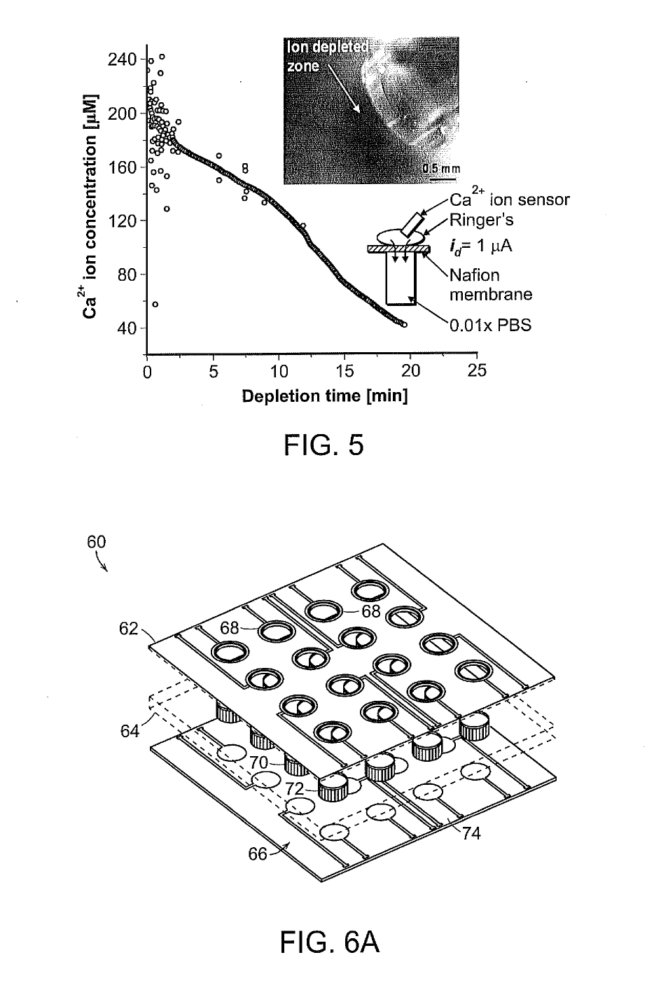

The depletion performance of an ion-selective electrode in-situ is characterized. Using a Ca²⁺ ion sensitive sensor, one could measure the depletion of Ca²⁺ ions as a function of time, as shown in FIG. 5. For this measurement, one can use a 200 μm thick Nafion membrane on a tip. Within 5 min., one could already decrease the Ca²⁺ ion concentration in Ringer's solution by ˜40 mM.

The flexible ion-selective microelectrode array device 2 is formed using standard microfabrication technique. The device has no ion reservoirs compared to the one based on the micropipette shown in FIG. 2A. There have already been several approaches reported on the ion-selective microelectrode arrays using the standard microfabrication techniques. All devices, however, have been fabricated exclusively in silicon material which requires an extensive fabrication in the cleanroom and therefore, a major cost factor in the commercialization efforts of the device. The flexible ion-selective microelectrode array device 2 includes PDMS (polydimethylsiloxane)/PI (polyimide) combination, common biocompatible elastomeric and thermoplastic materials used in BioMEMS. This material combination has several technological benefits compared to the standard silicon material. First, it is easily replicable and allows a mass manufacturing. Secondly, it is flexible and stretchable and can be easily bent to conform to the anatomical spatial constraints. Moreover, this type of flexible microelectrode array device 2 on polyimide substrate has been proven to be effective and biocompatible in-vivo application. However, no microelectrode array device 2 has been tested in-vivo with an integrated ion-selective membrane.

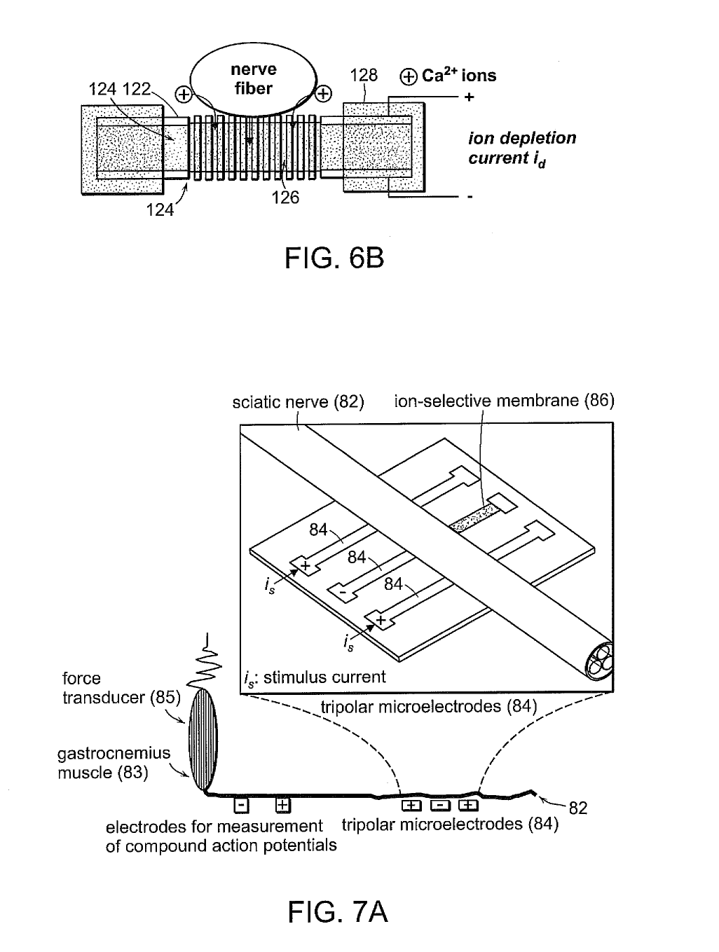

FIG. 6A shows a detailed view of a flexible ion-selective microelectrode array device 60 on flexible substrates in a sandwich design having three different layers. The flexible ion-selective microelectrode array device 60 includes three layers 62, 64, 66. The first layer 62 contains printed electrode array on an ultrathin polyimide layer (˜1 μm) for standard electrical stimulation. The metal electrodes in Au will be transferred on the polyimide layer via transfer printing method. A uniform layer of SiO₂ (˜50 nm) can be deposited by PECVD to form a passivation layer and etched away only at the ring-type electrodes 68. The second layer 64 is built out of PDMS with a thickness of 200 μm and contains holes with a diameter of 100 μm filled with ion-selective membranes 70, 72. The size of the hole up to 500 μm can be increased. The microspotting technique is used to fill the holes with either valinomycin for K⁺ or calcium ionophore II for Ca²⁺ ions and cure them at ambient temperature. If the microspotting technique doesn't deliver a satisfactory result in terms of the membrane quality, integrated microchannels can be integrated into the PDMS layer 64 and use the capillary force to fill the holes with the ion-selective resins.

Directly underneath the PDMS layer 64, a third layer 66 is boned out of polyimide with an array of the electrodes 74 used for modulating the ion concentration in combination with the membranes 70, 72. The same transfer printing technique is used as for the first layer 62 to pattern the gold electrodes on this flexible substrate and deposit Cr (˜3 nm)/SiO₂ (˜30 nm) layer for a subsequent plasma bonding with the middle PDMS layer 64. As an alternative to the circular-type electrode, we can pattern micro holes on the polyimde layer via photolithography before Au deposition. In this way, we can create a “porous electrode” with a uniform pore size between 1-30 μm. The entire electrode array device 60 can be connected to external electronic circuitry via stud-ball bond technique for an electrode pitch of 1-2 mm. To improve the stability of the connection and to prevent short circuits, parylene C will be deposited around the connection pads. The major risk factor in the fabrication process is how strong the adhesion between PDMS and the ion-selective resin would be after filling and curing.

Eventually, the PDMS surface needs to be treated with silane before filling with the ion-selective resins. Also, the structural integrity of the ion-selective membranes has to be tested in unfolded and folded configuration. To characterize the in-vitro properties, the electrode array can be put into a Ringer's solution at room temperature and the standard impedance spectrum will be measured. In addition, pulse tests by applying biphasic charge balanced rectangular constant current pulses will be performed to estimate electrochemical durability of the electrodes. Once successfully fabricated, this flexible ion-selective microelectrode array device will be tested on nerves in-vitro and in-vivo environments. Especially, the in-vivo test inside a frog body will show us whether the device packaging is appropriate for an implant.

As an alternative to the microfabricated electrodes on a polyimide layer, one can also use a track-etched polycarbonate membrane or porous nylon membrane 124 with a pore 126 size between 1-30 μm, as shown in FIG. 6B. To fabricate an electrically conductive layer 122, a 1-50 nm thin ITO (indium-tin-oxide) or other electrically conductive layer can be deposited on the membrane surface via sputtering. The membrane 126 and conductive layer 122 are encapsulated in a PDMS layer 128,

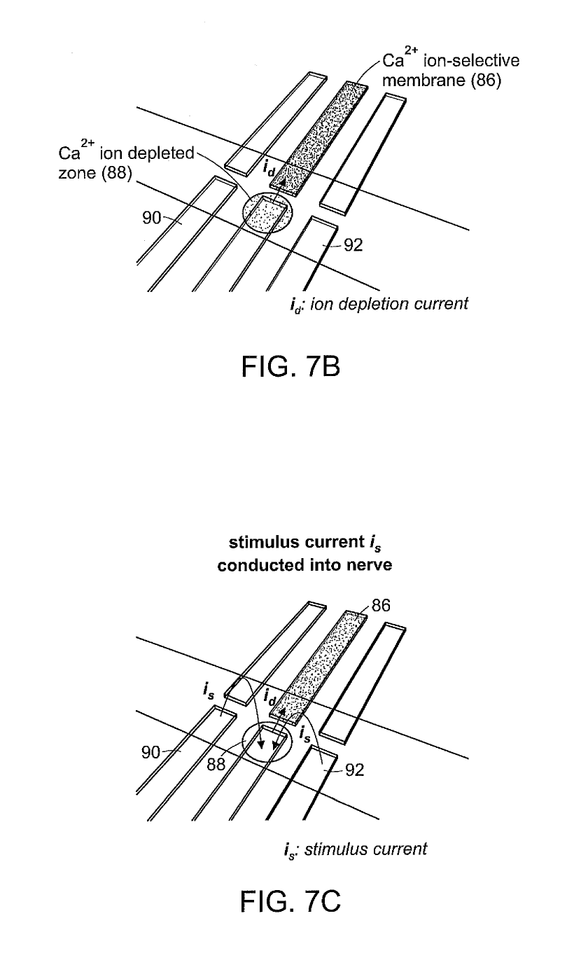

In another embodiment of the invention, one can place a sciatic nerve 82 on a microfabricated planar gold electrode array 80 without separating the perineurium while including the epineurium and stimulated the nerve electrically. This device has a planar design without ion reservoir having a single layer, as compared to the three-layer device in a sandwich design without ion reservoir in FIG. 6A. A schematic of the embodiment is shown in FIG. 7A with a top view of the planar electrode array 80. First, an ion depletion current i_{d} is applied across the ion-selective membrane 86 between two diametrically opposite electrodes 90, 92 in the center 88, as shown schematically in FIG. 7B. The ion depletion current i_{d} is limited to ≦1 μA, which was well below nominal current thresholds for electrical stimulation. The planar microelectrode covered with the ion-selective membrane 86 acted as a cathode and the opposite electrode 88 as an anode to deplete the positively charged Ca²⁺ ions. After depleting the ions at a given current i_{d} (ion depletion current i_{d} should be smaller than the threshold value for electrical stimulation iₛ) between the electrodes at a distance of 200 μm for duration t, an electrical stimulus current iₛ is applied between the two outer stimulating electrodes 90, 92 and the center contact electrode 88 (not covered with the Ca²⁺ ion-selective membrane 86) while i_{d} across the ion-selective membrane 86 was continuously applied. This tripolar electrode configuration 84 reduces the threshold current required to stimulate the nerve 82 and reduces the spread of the stimulus current along the nerve 82. Stimulation threshold currents, as well as resulting muscle contraction force originating from the stimulation were simultaneously measured and compared between conditions. The nerve 82 is coupled to the gastrocnemius muscle 83, which is coupled to a force transducer 85.

The planar microelectrodes were fabricated using the standard lift-off process. In brief, a 1 μm thick positive photoresist spin-coated on a 1 mm thick glass wafer is patterned photolithographically. After depositing a 50 nm Ti and 200 nm Au layer on the patterned glass wafer using the e-beam deposition, the photoresist layer was removed in acetone overnight. Before depositing an ion-selective membrane, the electrode was dehydrated at 90° C. on a hotplate for 24 h and then silanized with N,N-dimethyltrimethylsilylamine for 60 min. To deposit an ion-selective membrane a polydymethylsiloxane (PDMS) microchip is placed with a single microfluidic channel (300-1500 μm wide and 50 μm deep) and sealed it against the planar electrode after an optical alignment using a stereomicroscope. The ion-selective membrane for each specific ion was made using commercially available ion-selective cocktails, potassium ionophore I for K⁺ ion, sodium ionophore I for Na⁺ ion and ETH124 (calcium ionophore II) for Ca²⁺ ion, in a plasticized amorphous polymer matrix such as polyvinyl chloride (PVC).

Using the capillary force, the ion-selective resin mixture (10 wt. % for Ca²⁺ ionophore, 20 wt. % for K⁺ and Na⁺ ionophores in a plasticized amorphous matrix consisting of 35.8 mg polyvinyl chloride in 0.4 mL cyclohexanone) was filled into the microchannel. The PDMS channel was immediately removed once the electrode has been covered with the ion-selective resin and the electrodes were stored in a darkroom and dried for 12 hours under ambient conditions. To deposit cation-selective membrane on the planar electrodes, Nafion perfluorinated resin solution is used with 20 wt. % in mixture of lower aliphatic alcohols and water.

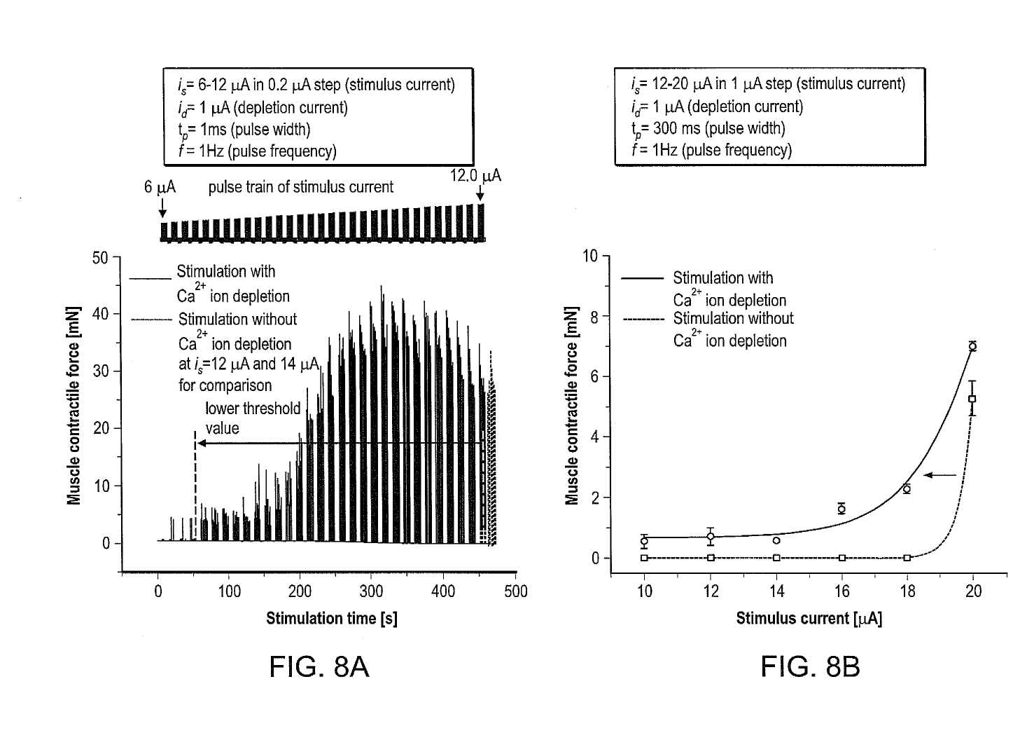

In all tests, the electrical current stimulation threshold is first measured without ion depletion or modulation. While there was a variation between different animal preparations, a pulse train is used between iₛ=4 and 20 μA at a pulse width of tₚ=300 μs or 1 ms and a pulse frequency of f=1 Hz. Using a microfabricated Ca²⁺ ion-selective membrane on the sciatic nerve of a frog, a decrease of the electrical threshold value is achieved from 12 μA down to 6.8 μA by approximately 40%, as shown in FIG. 8A. To deplete the Ca²⁺ ions, a depletion current of i_{d}=1 μA is first applied across a Ca²⁺ ion-selective membrane for 5 min. and then applied a stimulation electrical pulse iₛ directly thereafter. As a control experiment to verify the effect of Ca²⁺ ion depletion on the nerve excitability, the ion-selective membranes are integrated into a glass pipette tip and observed a continuous decrease of the electrical threshold value from 20 μA down to 10 μA by 50%, as shown in FIG. 8B. Slightly above the reduced stimulation threshold, the muscle twitch force amplitude was attenuated by approximately 90%, gradually increasing with increased stimulation current afterwards.

This observation is a qualitatively different behavior from common “all-or-none” electrical stimulation. This result clearly implies that the activity of muscle in terms of force can be controlled with higher degree of resolution and dynamic range, compared with the case of purely electrical stimulation. Even under a constant perfusion of Ringer's solution onto the nerve at the site of stimulation with a flow rate of 0.5 μL/min, which aimed at emulating the in-vivo ion homeostasis conditions, one could lower the electrical threshold from iₛ=5.6 to 4.4 μA. Under a constant perfusion of Ringer's solution on the stimulation site with the depletion current turned off, the original nerve excitability state was restored, both in terms of the current stimulation threshold and the characteristically sharp transition between “all-or-none” force generation. As a negative control experiment, the same stimulation test is performed with a plasticized amorphous polymer matrix such as PVC (polyvinyl chloride) membrane in a glass pipette tip without Ca²⁺ ion-specific ionophore conditions and confirmed that the electrical threshold value remained the same under continuous perfusion of Ringer's solution.

The in-vitro experimental results using a microfabricated planar ISM as well as a conventional ISM in the form of a glass pipette tip demonstrate that the depletion of Ca²⁺ ions can reduce the electrical threshold value by approximately 40% without a constant perfusion and approximately 20% under a constant perfusion of the Ringer's solution. In the case of the microfabricated ISM, one can demonstrated that a thin ion-selective membrane layer deposited on a planar microelectrode can be used as a selective ion reservoir to deplete and store the target ion from a zone adjacent the nerve by controlling the potential/current across the ISM.

A local in-situ control of ion concentration has been utilized to achieve higher excitable states for electrical stimulation. This significant reduction of the electrical threshold value could be achieved at a depletion current of i_{d}≦1 μA (usually less than 2V applied across the ion-selective membrane to maintain the ion depletion current in the microfabricated electrodes), and the power expenditure expected for the ion depletion was approximately 2 μW. It is likely that one can increase the efficacy of this method (in terms of speed and threshold reduction) by utilizing higher ion depletion currents.

However, water is hydrolyzed at electrode potentials over approximately 2V and above this voltage chlorine ions can be oxidized at the electrode surface to produce toxic compounds which set a limit to the applicable potential. To overcome this limitation, one could further decrease the gap size between the electrodes (currently 200 μm). The ability of ion-selective membranes to change the ion concentration depends on both the amount of ions adjacent to the nerve and the reservoir capacity of the ISM to store specific ion species. To maximize the amount of ions stored in the ISM, one can plan to optimize the geometry of the ISM in terms of width and thickness as well as the amount of ionophores in the ISM. The porosity and the pore size of the ISM are other important parameters to take into consideration. The reservoir ISM may be emptied to the vicienity by switching the polarity of the depleting or concentrating electrodes or by using another electrode that may be coupled with the electrode covered by the ISM.

As confirmed herein, the role of Ca²⁺ ions in nerve excitation in a separate control experiment where the nerve was completely immersed in a Ca²⁺ ion depleted Ringer's bath solution. In this context, an important point to consider is whether the isotonic Ringer's solution used in the in-vitro experiment is representative for the extracellular fluid in-vivo. Ringer's solution as an isotonic solution with a similar ionic composition to that of the extracellular fluid is widely used in the study of peripheral nerve excitability. The fact that the perineurium acts as a diffusion barrier to proteins and small molecules and thereby reduces the influence of proteins and molecules on nerve excitability also supports the use of Ringer's solution in our experiments. The only difference of using the extracellular fluid versus the Ringer's solution is that the presence of proteins and other molecules might have an impact on the lifetime of the ion-selective membranes due to non-specific binding. Furthermore, it has been demonstrated that the force amplitude generated at the downstream muscle can be more accurately controlled by the Ca²⁺ ion depletion. This result implies that a control of the contraction of muscle is possible with a higher degree of resolution and/or dynamic range than with traditional FES methods. It is hypothesized that the graded response of downstream muscle contraction may be due to the local manner of perturbing ion concentration (ion concentration of only one side of a fiber modulated).

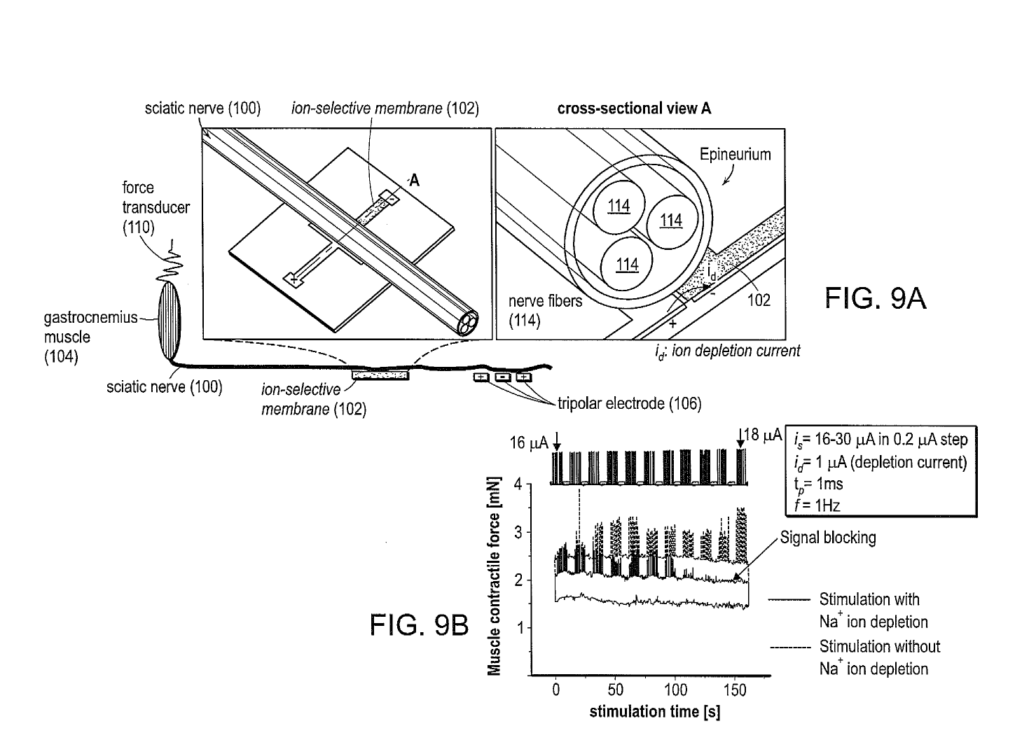

To investigate whether a modulation of the ion concentration along the nerve is an effective way of blocking the nerve signal conduction, a pair of 10 mm long and 750 μm wide Na⁺ ion-selective planar microelectrodes with a gap size of 300 μm are positioned between the site of electrical stimulation and the muscle 104, as shown in FIG. 9A. The sciatic nerve 100 is placed on top of a planar microelectrode deposited with the Na⁺ ion-selective membrane 102, as shown in the cross-sectional view A. The sciatic nerve 100 includes a number of nerve fibers 114. With this Na⁺ ion-selective membrane 102, a graded blocking of the sciatic nerve 100 for an electrical stimulus by depleting the Na⁺ ions from the nerve 100 and its surroundings is achieved without continuous perfusion of Ringer's solution, as shown in FIG. 9B. No muscle response was recorded at the force transducer 110 even at higher electrical pulses with iₛ over 30 μA after depleting Na⁺ ions for 5 min. with i_{d}=1 μA across the Na⁺ ion-selective membrane 102. To investigate the reversibility of the signal blocking method by the Na⁺ ion depletion, the ion depletion current is turned off and immersed the nerve with Ringer's solution. Recovery to the previous nerve excitability state lasted approximately 10 minutes in Ringer's solution.

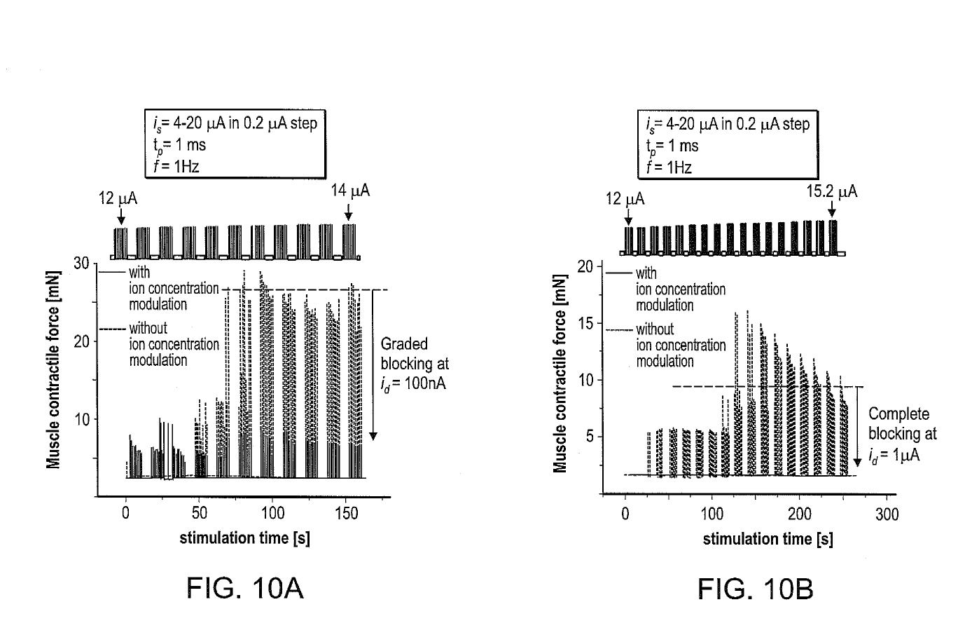

It is also observed a similar blocking effect when modulating the K⁺ ion concentration with a K⁺ ion-selective pipette tip. It seemed that injecting K⁺ ions from the pipette tip onto the nerve was more effective in terms of nerve signal blocking that depleting these ions under continuous perfusion of Ringer's solution. When using a cation-selective membrane such as Nafion with a reversed polarity of the ISM (the Nafion membrane deposited on the anodic side), which creates a general ion depletion zone (depletes all ions), a similar blocking effect is achieved as shown in FIGS. 10A-10B.

Using different ion depletion currents i_{d} at the same depletion time t=5 min., the blocking state is modulated from a partial at i_{d}=100 nA, as shown in FIG. 10A, to a complete blocking at i_{d}=1 μA, as shown in FIG. 10B. The blocking state was strongly dependent on the length of the ion-depleted zone, since there is a threshold length of the nerve required to be affected in order to achieve effective suppression. The nerve blocking could be obtained only when the length of the ion-selective membrane was 10 mm. In the case of a 200 μm long electrode, such as the one used for stimulation in FIG. 7A, no blocking was achieved at all. More importantly, this nerve blocking state could be reversed by immersing the nerve in a bath of Ringer's solution for 10 min.

This cycle of inhibition and relaxation is repeated three times with an immersion of the nerve in Ringer's solution for 10 min. between each cycle. In addition to Nafion, one can potentially achieve a similar effect with other cation-selective membrane materials such as poly(3,4-ethylenedioxythiophene) doped with polystyrene sulphonate) (PEDOT:PSS) which, as an electrically conducting organic polymer, was previously used to demonstrate electronic control of the ion homeostasis in neurons.

Many neurological disorders are characterized by undesirable nerve activity, leading to unwanted sensation or muscle activity. If the action potentials propagating through the nerve could be blocked in a graded fashion, which has been demonstrated for the motor activity in this current study, the disabling condition could be alleviated or eliminated. An effective and reversible nerve conduction block would have significant clinical applications such as blocking chronic peripheral pain and halting involuntary motor activity, such as muscle spasms, spasticity, tics and choreas.

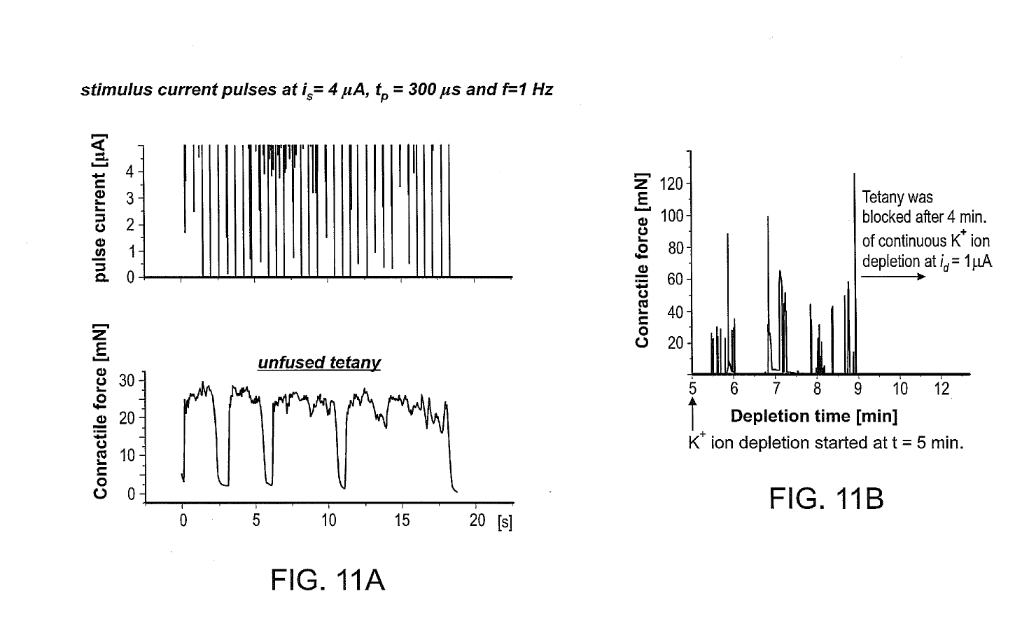

It is observed that a continuous depletion of Ca²⁺ ions can also cause a tetanic motion of the muscle. This type of motion is usually observed when the muscle is depleted of Ca²⁺ ions. As shown in FIG. 11A, this motion is observed by depleting the Ca²⁺ ions directly on the nerve. The stimulation current applied was simply too low to elicit a muscle contraction. In lower calcium levels (below 0.3 mM), alpha fibers in the sciatic nerve of frogs may become spontaneously active, leading to a tetanic-like situation. This unfused tetanic motion of the gastrocnemius muscle was completely reversible and could be stopped by applying Ringer's solution on the nerve. Alternatively, by depleting K⁺ ions with an ion-selective pipette tip, it was also possible to bring the uncontrolled tetanic motion, elicited by Ca²⁺ ion depletion, to a complete halt after 4 min. of K⁺ ion depletion, as shown in FIG. 11B. This finding may simulate the blockage of unwanted spasticity of the neuromuscular unit in a pathologic state.

One key constraint that could limit the effectiveness of our approach is the permeability of the perineurium for ions. The perineurium forms a continuous multilayered sheath around the fascicles of peripheral nerves and these morphological features contribute to the diffusion barrier properties of the perineurium to electron-dense tracers as well as to small ions. The limited permeability of the perineurium of the nerve creates a lag in the response of the nerve to the change of ion concentrations in the extracellular fluid. Also, depending on the diameter of the nerve, the majority of axons could be insensitive to the local ionic manipulation.

In order to translate these ideas to various neural prosthetic devices, longer-term, in-vivo reliability and safety studies need to be performed. Electrochemical reduction/oxidation processes at the electrodes, as well as any pH changes at the ion-selective membranes could be undesirable since they may alter the chemical composition of the extracellular fluid, producing cytotoxic compounds and effects. The pH shift at a current density of 10 μA/mm² seems to be of lesser concern for the experiments since the Ringer's solution was adequately buffered. In our ion-selective membranes, since the current density was significantly lower with ≦318 nA/mm² for the ion-selective pipette electrode and ˜5 μA/mm² for the planar ion-selective electrodes, one can also expect fewer problems with pH shift.

The invention demonstrates a novel means of using ion-selective membranes in modulating the activation and inhibition of nerve impulses in a reversible, graded fashion. These findings have potentially significant implications for the design of low-power, compact, neural prosthetic devices that selectively enhance nerve action potentials or inhibit unwanted motor endplate action potentials or noxious nerve stimulation. The devices demonstrated herein are readily applicable as electrochemical nerve manipulation technology, entirely controlled electrically without the need for chemical (ion) reservoirs and other complicated setup. These types of electrodes can be fabricated on a flexible substrate without any modification, for better enmeshing and contouring for nerve fibers and cells with various shapes and sizes.

In the invention, the ion depletion time could be significantly reduced because of increased surface contact area between the nerve and electrodes. With a projected flexible electrode system wrapped around the nerve, it is expected that one could achieve even higher control of nerve excitability. Finally, given the broad roles of ions such as Ca²⁺ in cellular signaling, the use of ion selective membranes demonstrated can be applied in other applications to directly control the important ionic species near the biological tissues and cells.

Although the present invention has been shown and described with respect to several preferred embodiments thereof, various changes, omissions and additions to the form and detail thereof, may be made therein, without departing from the spirit and scope of the invention.

What is claimed is:

1. A neural prosthetic device comprising one or more ion-selective membranes enabling by electrically-controlled local modulation of ion concentrations around a nerve so as to achieve different excitability states of the nerve for electrical stimulation or inhibition of nerve signal propagation.

2. The neural prosthetic device of claim 1, wherein the one or more ion-selective membranes receive a current to the ion concentrations around the nerve.

3. The neural prosthetic device of claim 1, wherein the one or more ion-selective membranes are positioned on a cathode structure.

4. The neural prosthetic device of claim 1, wherein the one or more ion-selective membranes modulate calcium ions to produce enhanced electrical stimulation.

5. The neural prosthetic device of claim 1, wherein the one or more ion-selective membranes modulate sodium or potassium ions to produce inhibition of nerve signal propagation.

6. The neural prosthetic device of claim 1, wherein the one or more ion-selective membranes comprise a plurality of ion-selective membranes arranged in a planar or sandwich configuration relative to a plurality electrodes to form an array of ion-selective microelectrodes.

7. The neural prosthetic device of claim 1, wherein the one or more ion-selective membranes are integrated into an electrode in the same plane or positioned between two electrodes.

8. The neural prosthetic device of claim 7, wherein the two electrodes comprise a photopatterned polymer layer with an array of microholes.

9. The neural prosthetic device of claim 8, wherein the two electrodes comprise one or more conductive layers having porous membranes with pore sizes of 1-30 μm.

10. The neural prosthetic device of claim 6, wherein the array comprises biocompatible materials.

11. The neural prosthetic device of claim 1, wherein the one or more ion-selective membranes are arranged in a bipolar or tripolar electrode arrangement.

12. The neural prosthetic device of claim 11, wherein the bipolar or tripolar electrode arrangement comprises a depletion zone for depleting ion concentrations that is induced by depletion current.

13. The neural prosthetic device of claim 11, wherein the bipolar or tripolar electrode arrangement comprises at two electrodes to induce stimulation of the ion concentrations in the one or more ion-selective membranes by inducing stimulation current.

14. A method of performing active nerve stimulation or inhibition of nerve signal propagation comprising:
providing one or more ion-selective membranes;
electrically controlling local modulation of ion concentrations using the one or more ion-selective membranes around a nerve so as to achieve different excitability states of the nerve for electrical stimulation or inhibition of nerve signal propagation.

15. The method of claim 14, wherein the one or more ion-selective membranes receive a current to the ion concentrations around the nerve.

16. The method of claim 14, wherein the one or more ion-selective membranes are positioned on a cathode structure.

17. The method of claim 14, wherein the one or more ion-selective membranes modulate calcium ions to produce enhanced electrical stimulation.

18. The method of claim 14, wherein the one or more ion-selective membranes modulate sodium or potassium ions to produce inhibition of nerve signal propagation.

19. The method of claim 14, wherein the one or more ion-selective membranes comprise a plurality of ion-selective membranes arranged in a planar or sandwich configuration relative to a plurality electrodes to form an array of ion-selective microelectrodes.

20. The method of claim 14, wherein the one or more ion-selective membranes are integrated into an electrode in the same plane or positioned between two electrodes.

21. The method of claim 20, wherein the two electrodes comprise a photopatterned polymer layer with an array of microholes.

22. The method of claim 21, wherein the two electrodes comprise one or more conductive layers having porous membranes with pore sizes of 1-30 μm.

23. The method of claim 19, wherein the array comprises biocompatible materials.

24. The method of claim 14, wherein the one or more ion-selective membranes are arranged in a bipolar or tripolar electrode arrangement.

25. The method of claim 24, wherein the bipolar or tripolar electrode arrangement comprises a depletion zone for depleting ion concentrations that is induced by depletion current.

26. The method of claim 24, wherein the bipolar or tripolar electrode arrangement comprises at two electrodes to induce stimulation of the ion concentrations in the one or more ion-selective membranes by inducing stimulation current.
